(19) United States
(12) Patent Application Publication
(10) Pub. No.: US 2015/0300977 A1
(43) Pub. Date: Oct. 22, 2015

(54) GLUCOSE SENSOR EMPLOYING SEMICONDUCTOR NANOELECTRONIC DEVICE

(71) Applicant: Trustees of Boston University, Boston, MA (US)

(72) Inventors: Pritiraj Mohanty, Los Angeles, CA (US); Shyamsunder Erramilli, Quincy, MA (US); Xihua Wang, Allston, MA (US); Yu Chen, Boston, MA (US)

(73) Assignee: Trustees of Boston University, Boston, MA (US)

(21) Appl. No.: 14/574,862

(22) Filed: Dec. 18, 2014

Related U.S. Application Data
(63) Continuation of application No. 12/894,792, filed on Sep. 30, 2010, now abandoned.
Application No. 12/894,792 is a continuation of application No. PCT/US2009/039087, filed on Apr. 1, 2009.
(60) Provisional application No. 61/072,716, filed on Apr. 1, 2008.

(51) Int. Cl. G01N 27/327; A61B 5/1486; A61M 5/172; A61B 5/145

(52) U.S. Cl. CPC G01N 27/3272; A61B 5/14532; A61B 5/14865; A61M 5/1723; A61B 2562/0285; A61M 2230/201; A61M 2205/3303

(57) ABSTRACT

A glucose sensor employs a programmable glucose sensor array of a relatively large number of nanoelectronic devices (e.g. semiconductor field-effect devices) having control surfaces functionalized with a glucose-reactive substance and generating sensing signals indicative of sensed glucose level of a bodily fluid. The devices arc divided into sub-sets sequentially enabled over successive intervals to achieve overall sensor lifetime many times longer than the lifetime of any single device in operation.

### BACKGROUND

The present invention is related to the field of blood glucose sensors and sensor/control systems.

Currently, blood glucose detection is mostly limited to in vitro testing of blood samples using enzyme based recognition. There is a medical need for performing in vivo testing by implantable glucose sensing devices for continued monitoring of the blood glucose level. Traditional glucose detectors are not suitable for such applications.

There is increasing interest in the use of nanoscale electronic devices for various sensing applications including blood glucose sensing. International patent publication WO 2008/063901A1 of Yu Chen et al. describes a nanochannel-based sensor system which may be used in a variety of sensing applications including blood glucose sensing. The sensor system employs an array of field-effect nanoelectronic devices having critical dimensions on the order of 100 nm or less, with surface functionalization to interact with a species of interest (such as the enzyme glucose oxidase to functionally interact with glucose in solution). Due to their nanoscale dimensions, the devices exhibit strong sensitivity to variations in surface charge arising from the functional chemical interaction, enabling sensitive detection of glucose levels. Glucose sensors using nanoscale electrical transducers provide a solution towards minimizing device size for implantable device applications, while also reducing device cost. Also, when a so-called “top-down” semiconductor manufacturing approach is used, additional benefits can be obtained including easier integration with supporting electronics and scalable manufacturing.

### SUMMARY

While nanoelectronic sensors display promise as glucose sensors, there remain certain challenges to any widespread use of this type. One significant challenge is presented by a relatively short useful lifetime of the devices when continuously in use. It has been observed that nanoelectronic sensors used in continual sensing of glucose in solution have a useful lifetime on the order of several days, after which their electrical response has diminished to an unacceptable level. It would be much more desirable for in-vivo applications for a sensor to function significantly longer once implanted or otherwise put into use by a user.

In the present disclosure, a glucose sensor employs a programmable glucose sensor array based on a set of semiconductor nanoelectronic devices (which can be fabricated using CMOS-compatible fabrication process) as the electrical transducer of the sensor. Because of the higher surface to volume ratio of the semiconductor nanostructures, electrical properties of the device are extremely sensitive to the surface potential, or surface charge change of these structures due to field effect. When the surface of these structures is functionalized with a glucose-reactive substance such as glucose oxidase, the device shows electrical signals when it comes in contact with blood samples containing glucose. Fabrication of semiconductor nanostructures as the electrical transducer will be helpful to minimize the sensor size and reduce the sensor cost. Construction of nanoscale electrical transducer benefits glucose sensor with all kinds of forms, including in vitro test and in vivo blood glucose level monitoring.

In particular, the sensor employs a generally large number of devices divided into sub-sets and sequentially enables different sub-sets of the devices over successive periods of operation in order to achieve overall sensor lifetime that is many times longer than the lifetime of any single device in operation. Because the devices degrade primarily during operation (and generally not during non-use even when exposed to body fluids such as blood), only the sub-sets of devices actually in use at a given time are actively degrading. Thus each sub-set is maintained inactive until it is selected, and all the sub-sets have about the same operating lifetime regardless of when activated. If a sensor has 10,000 devices for example and uses them in sub-sets of 10 at a rate of one sub-set each three days, the sensor may have a maximum lifetime on the order of 3,000 days.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 is a block diagram of a glucose sensor;

FIG. 2 (consisting of parts 2(a)-2(b)) depicts a nanochannel-based sensing clement in the glucose sensor of FIG. 1; and

FIG. 3 is a block diagram of a system mimicking operation of an animal pancreas for continually monitoring and controlling blood glucose level.

### DETAILED DESCRIPTION

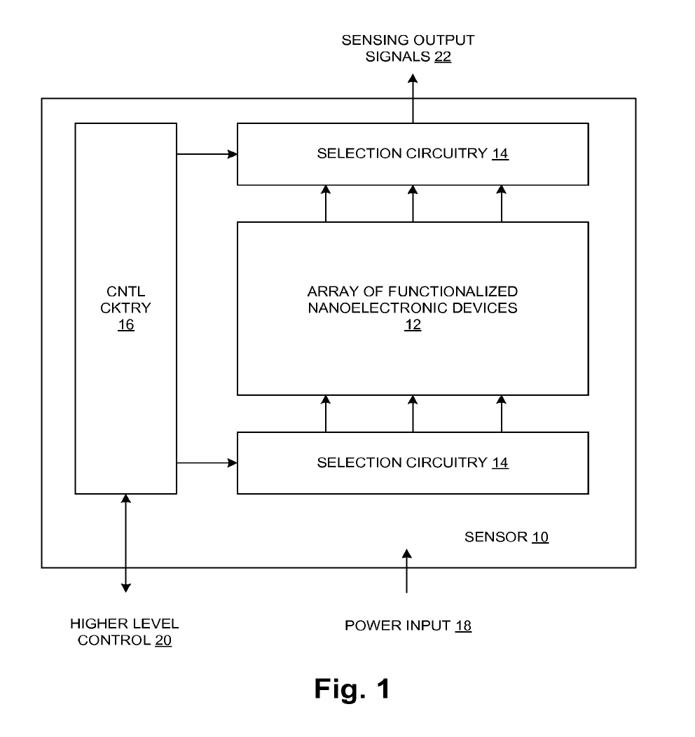

FIG. 1 shows a glucose sensor 10 which includes an array of functionalized nanoelectronic devices 12, selection circuitry 14 and control circuitry 16. The sensor 10 receives operating power via a power input 18 and includes an interface to external higher-level control 20 as well as sensing output signals 22 which correspond to glucose concentration levels as sensed by active devices within the array 12. Details of the array 12 are discussed below, as well as applications/uses of the sensor 10 which involve the various interfaces/signals 18-22.

The array 12 includes a relatively large number of individual nanoelectronic devices, arranged to be selectively activated by the selection circuitry 14 in response to control signals from the control circuitry 16. The unit of activation is herein referred to as a “subset”, and may range from as few as one to perhaps 10 or more devices, depending on a variety of factors including signal-to-noise considerations, reliability, need for control or reference devices in each subset for greater accuracy/precision, etc. In one class of embodiments each subset has in the range of 3 to 10 devices. The overall number of devices may vary widely in different embodiments, from as few as 10 to over 10,000 for example, and will also depend on a variety of factors such as intended application and desired lifetime, cost, etc. Devices within the array 12 may be laid out in a linear fashion, or as a rectangular grid, or other arrangements as desired.

In use, the array 12 of the sensor 10 is exposed to a glucose-carrying fluid such as blood for example, and the devices of the currently active subset respond by assuming corresponding electrical conduction characteristics that become manifested as the sensing output signals 22 (which may be voltage and/or current signals whose values correspond to sensed glucose levels through the action of the active devices of the array 12). The sensor 10 may be implanted in a subject's body to be in contact with the glucose-carrying fluid, or in other uses the sensor 10 may be external to the subject's body and the glucose-carrying fluid is supplied to the sensor 10 in some manner. The sensor 10 preferably includes a fluid interface structure to channel the bodily fluid to the active surfaces of the devices of the array 12 (see description of devices below). The fluid interface structure could be a machined chamber integrated on top of the sensor (like PDMS or plastic chamber). It could be micromachined in the same wafer, which will contain the chamber (like a lab-on-a-chip) and the sensor (fabricated inside the chamber). The chamber can be designed to control the in and out flow of the fluid. The chamber volume could be less than 50 microliters, 100 microliters, 1 milliliter.

The control circuitry 16 and selection circuitry 14 operate together to systematically select successive new subsets of devices during device use in order to achieve an overall operating lifetime of the sensor 10 that is significantly longer than the useful operating lifetime of an individual device, which as noted above may be only on the order of a few days. In one type of embodiment, the control circuitry 16 causes the selection circuitry 14 to activate a new subset at regular predetermined intervals, such as once every three days for example. Such predetermined intervals may be fixed or programmable. As an alternative, the control circuitry 16 may employ some form of performance monitoring of the active subset and switch to a new subset only when the current subset shows sufficient operational degradation to signal the need for a switch. As an example, the control circuitry 16 may monitor for a certain percentage reduction in output levels under known conditions (relying for example on known good reference devices) to identify the need to switch to a new subset. Such performance monitoring could be used either instead of or in addition to the use of a regular predetermined interval.

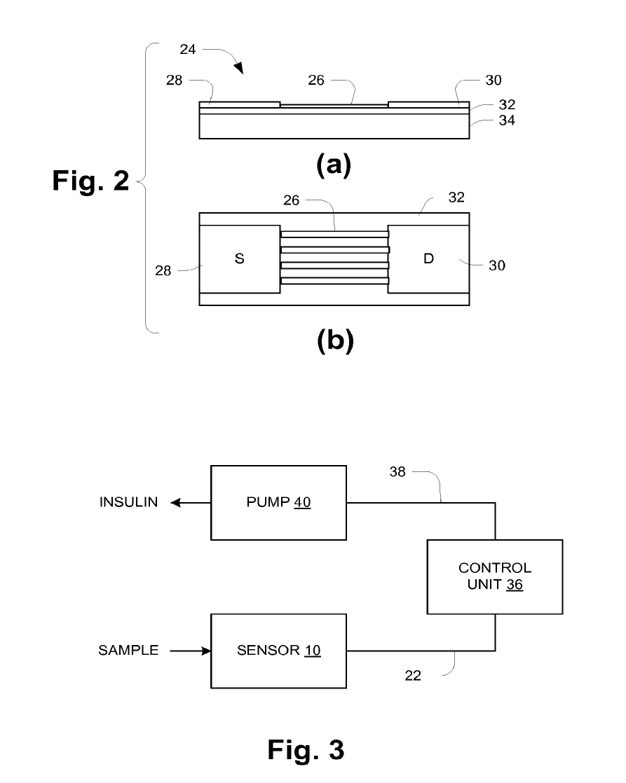

FIG. 2 shows an individual sensing element or device 24 according to one embodiment. As shown in the side view of FIG. 2(a), silicon nanochannels 26 extend between a source (S) contact 28 and a drain (D) contact 30, all formed on an insulating oxide layer 32 above a silicon substrate 34. FIG. 2(b) is a top view showing the narrow elongated nanochannels 26 extending between the wider source and drain contacts 28, 30 which are formed of a conductive material such as gold-plated titanium for example. In certain embodiments, each nanochannel 26 preferably includes an outer oxide layer such as aluminum oxide.

Thus in one embodiment the sensor 10 uses nanoelectronic devices 24 made of semiconductors, such as silicon, as the electrical transducer. Particularly silicon nanostructures, such as nanochannels, nanobelts, or nanowires, can be fabricated from a silicon-on-insulator (SOI) wafer. The SOI wafer consists of a device layer typically less than 200 nm thick, a silicon substrate, and an insulating layer of SiO2 in between. The nanoelectronic devices 24 can be patterned with electron beam lithography or photolithography, and all side walls are exposed after reactive ion etching (RIE) for increasing the surface-to-volume ratio. Metals, such as Ti/Au, are deposited with thermal evaporator or electron beam evaporator as the source and drain contact electrodes, without further annealing process. The nanochannels 26 are preferably on the order of 100 nm or less in width, and can be covered with an Al203 layer, grown by atomic layer deposition (ALD), with a typical thickness of 10 nm. The silicon top layer is lightly doped with boron with a concentration of 10-15 cm-3 as the device layer.

The signal according to glucose concentration in the test sample should refer to the electrical properties of the nanostructures. One example is that the differential conductance of the devices 24 in the array gives the glucose concentration. Another example is that the calibrated surface potential of the devices 24 shows the glucose concentration. Although not shown in FIG. 2, an additional side gate may be used to electrolyze hydrogen peroxide and increases the lifetime of the devices 24 in the array 12.

As shown in FIG. 2, an individual device 24 may include multiple nanochannels 26. In the illustrated embodiment the device 24 includes four nanochannels 26, but in alternative embodiments a single device 24 may have more or less. Although not specifically shown, a subset (the unit of activation) includes a plurality of individual devices 24. Techniques for individually activating a group or set of electronic devices are generally known and not elaborated herein.

Returning briefly to FIG. 1, during a given operating interval the control circuitry 16 may operate the devices 24 of the selected sub-set in a pulsed or sampled manner, providing power to the devices only at regular sample times rather than continually throughout the interval. By using such sampled operation of the nanoelectronic devices of the selected subset, reduced power consumption can be achieved compared to continuous operation of the nanoelectronic devices. This reduced power consumption can translate into increased lifetime of a limited-storage power supply (such as a battery) used to supply power to the sensor 10.

FIG. 3 shows an application of the glucose sensor 10 in a system including a control unit 36 and a pump 38, which can operate in a manner analogous to an animal pancreas to regulate blood glucose levels by selective release of the hormone insulin. The sensor 10 is exposed to a glucose-carrying bodily fluid (shown as SAMPLE in FIG. 3) and generates sensing output signals 22 which are provided to the control unit 36. The control unit 36 performs an appropriate control algorithm to ascertain an amount of insulin to be supplied based on the sensed glucose level as conveyed by the sensing output signals 22, and generates pump control signals 38 which are supplied to an insulin pump 40 which dispenses the insulin in accordance with the values of the pump control signals 38. The control unit 36 may also have a separate interface (not shown) to the sensor 10 to serve as the higher-level control 20 shown in FIG. 1.

While various embodiments of the invention have been particularly shown and described, it will be understood by those skilled in the art that various changes in form and details may be made therein without departing from the spirit and scope of the invention as defined by the appended claims.

For example, different variations of semiconductor nanostructures may be used as the electrical signal transducer. While silicon may be a desirable material for its compatibility with integrated circuits, other materials such GaAs can be used as the building material of the device. Within an array of such devices, it may be desirable to refrain from functionalizing some devices to enable them to serve as references. High density nanoscale electrical transducers can help to increase sensitivity by averaging all working elements in the array.

What is claimed is:

1. A glucose sensor, comprising:
a nanoelectronic device having a control surface functionalized with a glucose-reactive substance; and
a fluid interface structure configured to allow contact between the control surface and a bodily fluid.

2. A glucose sensor according to claim 1, comprising an array of the nanoelectronic devices having respective control surfaces also functionalized with the glucose-reactive substance, and wherein the fluid interface structure is configured to allow contact between the control surfaces and the bodily fluid.

3. A glucose sensor according to claim 1, wherein the nanoelectronic device is configured such that chemical interaction between the glucose-reactive substance and glucose in the bodily fluid affects electrical conduction characteristics of the nanoelectronic device.

4. A glucose sensor according to claim 1, wherein the nanoelectronic device is configured such that chemical interaction between the glucose-reactive substance and glucose in the bodily fluid effects capacitive or other parametric changes of the nanoelectronic device.

5. A glucose sensor according to claim 1, wherein the nanoelectronic device has a sensing element critical dimension less than 100 nm.

6. A glucose sensor according to claim 1, wherein the nanoelectronic device has a sensing element critical dimension less than 500 nm.

7. A glucose sensor, comprising:
an array of nanoelectronic devices having respective control surfaces functionalized with a glucose-reactive substance which chemically interacts with glucose to affect electrical conduction characteristics of the nanoelectronic devices, the array being configured to allow for intimate contact between the control surfaces and a glucose-carrying bodily fluid, the array of nanoelectronic devices being logically organized into a plurality of individually operable subsets of the nanoelectronic devices, each subset being operable for only a limited period before operational degradation due to interaction between the bodily fluid and operating nanoelectronic sensors of the subset;
device selection circuitry operative in response to control inputs to enable electrical sensing operation of a selected one of the subsets of the nanoelectronic devices to generate respective sensing output signals while simultaneously disabling such electrical sensing operation of remaining ones of the subsets of the nanoelectronic devices; and
control circuitry operative to generate the control signals so as to serially enable electrical operation of successive ones of the subsets of the nanoelectronic devices over an extended period generally equal to the product of the limited period and the number of the subsets of the nanoelectronic devices.

8. A glucose sensor according to claim 7 wherein the array is configured for implantation into a body tissue to provide for the intimate contact between the control surfaces and the glucose-carrying bodily fluid.

9. A glucose sensor according to claim 7 wherein the nanoelectronic sensors are nanoscale field-effect devices.

10. A glucose sensor according to claim 7 wherein the control circuitry is further operative to effect sampled operation of the nanoelectronic devices of the selected subset to achieve reduced power consumption compared to continuous operation of the nanoelectronic devices.

11. A glucose sensor according to claim 7 wherein the control circuitry is further operative to engage in performance monitoring of the nanoelectronic devices to ascertain how accurately the sensing output signals reflect an actual glucose level of the glucose-carrying bodily fluid.

12. A glucose sensor according to claim 11 wherein the performance monitoring is utilized to switch to a new subset when a current subset shows sufficient operational degradation to signal the need for a switch.

13. A glucose sensor according to claim 11 wherein predetermined ones of the nanoelectronic devices are operated as control devices whose outputs are utilized in the performance monitoring of the control circuitry.

14. A system for controlling blood glucose level by selective administration of insulin to a subject, comprising:
the glucose sensor of claim 1 having the control surface in intimate contact with the bodily fluid of a subject;
an insulin pump configured to administer insulin to the subject as a function of pump control signals supplied to the insulin pump; and
a control unit coupled to receive a sensing output signal from the glucose sensor and to perform a control algorithm to (1) ascertain an amount of insulin to be supplied to the subject based on sensed glucose levels as conveyed by the sensing output signal, and (2) generate the pump control signals to cause the insulin pump to dispense the ascertained amount of insulin.

15. A system according to claim 14 wherein the glucose sensor is implanted into a body tissue of the subject.

16. A method of continual, extended sensing of glucose level of a glucose-carrying bodily fluid, comprising:
bringing the glucose-carrying bodily fluid into intimate contact with control surfaces of an array of nanoelectronic devices of a glucose sensor, the control surfaces being functionalized with a glucose-reactive substance which chemically interacts with glucose to affect electrical conduction characteristics of the nanoelectronic devices, the array of nanoelectronic devices being logically organized into a plurality of individually operable subsets of the nanoelectronic devices, each subset being operable for only a limited period before operational degradation due to interaction between the bodily fluid and operating nanoelectronic sensors of the subset;
in response to control inputs, enabling electrical sensing operation of a selected one of the subsets of the nanoelectronic devices to generate respective sensing output signals while simultaneously disabling such electrical sensing operation of remaining ones of the subsets of the nanoelectronic devices; and
generating the control inputs to serially enable electrical operation of successive ones of the subsets of the nanoelectronic devices over an extended period generally equal to the product of the limited period and the number of the subsets of the nanoelectronic devices.

17. A method according to claim 16 further comprising operating the nanoelectronic devices of the selected subset in a sampled manner to achieve reduced power consumption compared to continuous operation of the nanoelectronic devices of the selected subset.

18. A method according to claim 16 further comprising engaging in performance monitoring of the nanoelectronic devices to ascertain how accurately the sensing output signals reflect an actual glucose level of the glucose-carrying bodily fluid.

19. A method according to claim 18 wherein the performance monitoring is utilized to switch to a new subset when a current subset shows sufficient operational degradation to signal the need for a switch.

20. A method according to claim 18 wherein predetermined ones of the nanoelectronic devices are operated as control devices whose outputs are utilized in the performance monitoring of the control circuitry.
